(19) United States
(12) Patent Application Publication
(10) Pub. No.: US 2006/0025721 A1
(43) Pub. Date: Feb. 2, 2006

(54) Catheter and guidewire exchange system with improved catheter design

(76) Inventors: Niall Duffy, Tuam (IE); Noel Coyle, Craughwell (IE); David Quinn, Salthill (IE)

Correspondence Address: MEDTRONIC VASCULAR, INC.;IP LEGAL DEPARTMENT, 3576 UNOCAL PLACE, SANTA ROSA, CA, 95403, US

(21) Appl. No.: 10/903,802

(22) Filed: Jul. 30, 2004

(51) Int. Cl. A61M 5/178

(52) U.S. Cl. 604/164.12; 606/108

(57) ABSTRACT

An assembly for guiding a catheter includes a catheter, a catheter advancing and retracting apparatus, and a guidewire removal tool. The catheter comprises an elongate shaft having an exterior surface, a proximal end, and a distal end; a first lumen extending through the shaft and sized to receive a guidewire; and a guideway enabling transverse access from the shaft exterior surface to the first lumen. The catheter advancing and retracting apparatus comprises an elongate housing; an opening formed through the housing and adapted to receive the catheter; and a linear slot formed in the housing and adapted to be aligned with a portion of the catheter guideway. The guidewire removal tool comprises a substantially cylindrical main body sized to be receivable by the first lumen and having a chamfered leading edge adapted to raise the guidewire out of the first lumen through the guideway.

### TECHNICAL FIELD

The present invention generally relates to catheters used in the vascular system, and more particularly relates to systems for facilitating exchange of such catheters and associated guidewires, and for using such catheters and guidewires to access selected sites within a patient.

### BACKGROUND

Cardiovascular disease, including atherosclerosis, is a leading cause of death in the U.S. The medical community has developed a number of methods and devices for treating coronary heart disease, some of which are specifically designed to treat the complications resulting from atherosclerosis and other forms of coronary arterial narrowing.

One method for treating atherosclerosis and other forms of coronary narrowing is percutaneous transluminal coronary angioplasty, commonly referred to as “angioplasty” or “PTCA.” The objective in angioplasty is to enlarge the lumen of the affected coronary artery by radial hydraulic expansion. The procedure is accomplished by inflating a balloon of a balloon catheter within the narrowed lumen of coronary artery.

In addition to PTCA, catheters are used for delivery of stents or grafts, therapeutic drugs (such as anti-vaso-occlusion agents or tumor treatment drugs) and radiopaque agents for radiographic viewing. Other uses for such catheters are well known in the art.

The anatomy of coronary arteries varies widely from patient to patient. Often a patient's coronary arteries are irregularly shaped, highly tortuous and very narrow. The tortuous configuration of the arteries may present difficulties to the physician in proper placement of a guidewire, and advancement of a catheter to a treatment site. A highly tortuous coronary anatomy typically will present considerable resistance to advancement of the catheter over the guidewire.

Therefore, it is important for a catheter to be highly flexible. However, it is also important for a catheter shaft to be stiff enough to push the catheter into the vessel in a controlled manner from a position far away from the distalmost point of the catheter.

Catheters for PTCA and other procedures may include a proximal shaft, a transition section and a distal shaft having a flexible distal tip. In particular, the catheters have a proximal shaft, which is generally rigid for increased pushability and a more flexible distal shaft with a flexible distal tip for curving around particularly tortuous vessels. The proximal shaft may be made stiff by the insertion of a thin biocompatible tube, such as a stainless steel hypotube, into a lumen formed within the proximal shaft. The transition section is the portion of the catheter between the stiffer proximal shaft and the more flexible distal shaft, which provides a transition in flexibility between the two portions.

With some types of catheter construction, when an increase in resistance occurs during a procedure there is a tendency for portions of the catheter to collapse, buckle axially or kink, particularly in an area where flexibility of the catheter shaft shifts dramatically. Consequently, the transition section is often an area where the flexibility of the catheter gradually transitions between the stiff proximal shaft and the flexible distal shaft. It is known in the art to create a more gradual flexibility transition by spiral cutting a distal end of the hypotubing used to create stiffness in the proximal shaft. Typically, the spiral cut is longitudinally spaced father apart at the hypotube proximal end creating an area of flexibility, and longitudinally spaced closer together at the hypotube distal end creating an area of even greater flexibility.

In a typical PTCA procedure, it may be necessary to perform multiple dilatations, for example, using various sized balloons. In order to accomplish the multiple dilatations, the original catheter must be removed and a second catheter tracked to the treatment site. When catheter exchange is desired, it is advantageous to leave the guidewire in place while the first catheter is removed to properly track the second catheter.

Two types of catheters commonly used in angioplasty procedures are referred to as over-the-wire (OTW) catheters and rapid exchange (RX) catheters. A third type of catheter with preferred features of both OTW and RX catheters, which is sold under the trademarks MULTI-EXCHANGE, ZIPPER MX, ZIPPER, MX and/or MXII, is discussed below. An OTW catheter's guidewire lumen runs the entire length of the catheter and may be positioned next to, or enveloped within, an inflation shaft. Thus, the entire length of an OTW catheter is tracked over a guidewire during a PTCA procedure. A RX catheter, on the other hand, has a guidewire lumen that extends within only the distalmost portion of the catheter. Thus, during a PTCA procedure only the distalmost portion of a RX catheter is tracked over a guidewire.

If a catheter exchange is required while using a standard OTW catheter, the user must add an extension wire onto the proximal end of the guidewire to maintain control of the guidewire, slide the catheter off of the extended guidewire, slide the new catheter onto the guidewire and track back into position. Multiple operators are required to hold the extended guidewire in place while the original catheter is exchanged in order to maintain its sterility.

A RX catheter avoids the need for multiple operators when exchanging the catheter. With a rapid exchange catheter, the guidewire runs along the exterior of the catheter for all but the distalmost portion of the catheter. As such, the guidewire can be held in place without an extension when the catheter is removed from the body. However, one problem associated with RX catheters is the guidewire, and most of the catheter, must be removed from the body in order to exchange guidewires. Essentially the procedure must then start anew because both the guidewire and the catheter must be retracked to the treatment site. An OTW catheter, with the guidewire lumen extending the entire length of the catheter, allows for simple guidewire exchange.

A balloon catheter capable of both fast and simple guidewire and catheter exchange is particularly advantageous. A catheter designed to address this need is sold by Medtronic Vascular, Inc. of Santa Rosa, Calif. under the trademarks MULTI-EXCHANGE, ZIPPER MX, ZIPPER, MX and/or MXII (hereinafter referred to as the “MX catheter”). An MX catheter is disclosed in U.S. Pat. No. 4,988,356 to Crittenden et al.; co-pending U.S. patent application Ser. No. 10/116,234, filed Apr. 4, 2002; co-pending U.S. patent application Ser. No. 10/251,578, filed Sep. 18, 2002; co-pending U.S. patent application Ser. No. 10/251,477, filed Sep. 20, 2002; co-pending U.S. patent application Ser. No. 10/722,191, filed Nov. 24, 2003; and co-pending U.S. patent application Ser. No. 10/720,535, filed Nov. 24, 2003, all of which are incorporated by reference in their entirety herein.

The MX catheter includes a catheter shaft having a guidewire lumen positioned side-by-side with an inflation lumen. The MX catheter also includes a longitudinal cut that extends along the catheter shaft and that extends radially from the guidewire lumen to an exterior surface of a catheter shaft. A guide member through which the shaft is slidably coupled cooperates with the longitudinal cut such that a guidewire may extend transversely into or out of the guidewire lumen at any location along the longitudinal cut's length. By moving the shaft with respect to the guide member, the effective over-the-wire length of the MX catheter is adjustable.

The guidewire is threaded into a guidewire lumen opening at the distal end of the catheter and out through the guide member. The guidewire lumen envelopes the guidewire as the catheter is advanced into the patient's vasculature while the guide member and guidewire are held stationary. Furthermore, the indwelling catheter may be removed by withdrawing the catheter from the patient while holding the proximal end of the guidewire and the guide member in a fixed position. When the catheter has been withdrawn to the point where the distal end of the cut has reached the guide member, the distal portion of the catheter over the guidewire is of a sufficiently short length that the catheter may be drawn over the proximal end of the guidewire without releasing control of the guidewire or disturbing its position within the patient.

Recently efforts have been directed toward minimizing the complexity and size of the guide tool that is used with the MX catheter. The catheter with the side-by-side lumen arrangement has a relatively large outer diameter, and requires a correspondingly large guide tool to accommodate the catheter. Accordingly, it is desirable to provide an MX catheter that has a smaller outer diameter, so that the guide tool can have a correspondingly smaller inner diameter, and consequently a smaller overall size. In addition, it is desirable to provide a guide tool that is less complex than the current guide tool, allowing the user to quickly troubleshoot, prevent, and correct any difficulties that may occur during guide tool operation. Furthermore, other desirable features and characteristics of the present invention will become apparent from the subsequent detailed description and the appended claims, taken in conjunction with the accompanying drawings and the foregoing technical field and background.

### BRIEF SUMMARY

The present invention is directed to a catheter that comprises an elongate shaft having an exterior surface, a proximal end, and a distal end; a first lumen extending through the shaft from the shaft proximal end to the shaft distal end, and sized to receive a guidewire; and a guideway extending from the shaft proximal end to the shaft distal end, and enabling transverse access from the shaft exterior surface to the first lumen. The present invention is also directed to a catheter advancing and retracting apparatus that comprises an elongate housing having a proximal end and a distal end; an opening formed through the housing from the housing proximal end to the housing distal end and adapted to receive the catheter; and a linear slot formed in the housing proximal end and adapted to be aligned with a portion of the catheter guideway. The present invention is further directed to a guidewire removal tool that comprises a substantially cylindrical main body sized to be receivable by the first lumen and having a chamfered leading edge adapted to raise the guidewire out of the first lumen through the guideway.

### BRIEF DESCRIPTION OF THE DRAWINGS

The present invention will hereinafter be described in conjunction with the following drawing figures, wherein like numerals denote like elements, and

FIG. 1 is a perspective view of a guide member with a guide wire extending through the guide member and into a catheter according to the present invention;

FIG. 2 is a perspective view of a catheter guiding assembly according to an embodiment of the present invention;

FIG. 3 is a perspective view of a catheter advancement/retraction wheel according to an embodiment of the present invention;

FIG. 4 is a perspective view of the catheter advancement/retraction wheel frictionally engaged with a novel catheter according to an embodiment of the present invention;

FIG. 5 is a sectional view of a catheter guiding assembly including the catheter advancement/retraction wheel frictionally engaged with the catheter according to an embodiment of the present invention; and

FIG. 6 is a sectional view of a catheter according to an embodiment of the present invention, including a nearly coaxially arranged guidewire lumen and inflation lumen.

FIG. 7 is a sectional view of a catheter with a guidewire being removed from a guidewire lumen using a scoop according to an embodiment of the present invention;

FIG. 8 is a sectional view of another embodiment of a guidewire removal tool according to an embodiment of the present invention;

FIG. 9 is a sectional view of a catheter receiving a guidewire removal tool according to an embodiment of the present invention;

FIG. 10 is a perspective view of a guidewire removal tool according to an embodiment of the present invention;

FIG. 11 is a sectional view of a catheter with the guidewire removal tool depicted in FIG. 10 inserted into a guidewire lumen according to an embodiment of the present invention;

FIG. 12 is a perspective view of the guide member distal end in cooperation with the guidewire removal tool from FIGS. 10 and 11 and a catheter; and

FIG. 13 is a perspective view of the guide member distal end and the guidewire removal tool illustrated in FIG. 12, with the catheter removed to more clearly illustrate the relationship between the guide member and the tool.

### DETAILED DESCRIPTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

The present invention includes a catheter construction with a relatively small outer diameter, and tools for advancing and retracting the catheter and for coupling and decoupling the catheter with a guidewire. A particular feature of the present invention includes tools that are used to decouple the catheter and guidewire. The decoupling tool is attached to, or utilized near the proximal end of a catheter guiding assembly that advances and retracts a catheter with respect to a patient's body. For the purposes of clarity, the catheter and the catheter guiding assembly will be described generally, followed by a detailed description of various decoupling tools, also referred to as guidewire removal tools.

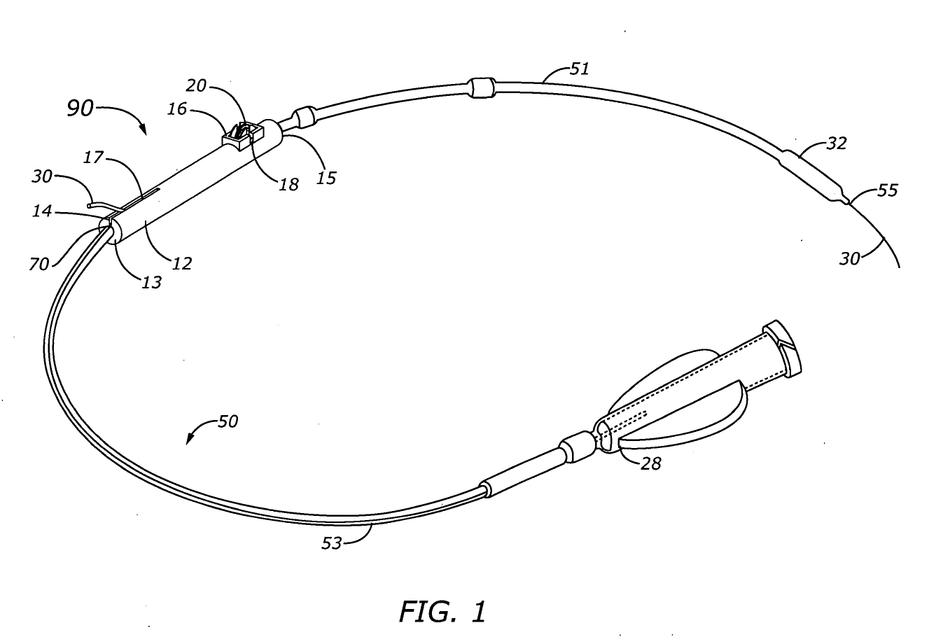

FIG. 1 is a perspective view of a catheter 50 including a catheter guiding assembly 90 that are useful with various decoupling tools according to an embodiment of the invention. The catheter 50 includes an elongate, flexible, cylindrical main body having a distal shaft 51 and a proximal shaft 53. According to the present embodiment, the catheter 50 is a delivery catheter for such procedures as PTCA or stent delivery and has a balloon 32 mounted around the catheter body near the catheter distal end 55. The balloon 32 may be inflated and deflated through the catheter inflation lumen 54.

Turning briefly to FIG. 6, a perspective view of a cross section of the catheter 50 illustrates an inflation lumen 54 and a guidewire lumen 58 as the catheter passageways. The inflation lumen communicates with a fitting 28 depicted in FIG. 1 at the catheter proximal end, and extends the catheter length to terminate in communication with the balloon interior approaching the catheter distal end 55. The guidewire lumen 58 receives a guidewire 30 and extends the entire catheter length. A guideway 52 extends into the guidewire lumen 58 along the length of most of the proximal shaft 53 and provides access for insertion or withdrawal of the guidewire 30. The distal shaft 51 does not include the guideway 52, so the guidewire lumen 58 and the inflation lumen 54 are coaxially arranged in the distal shaft 51.

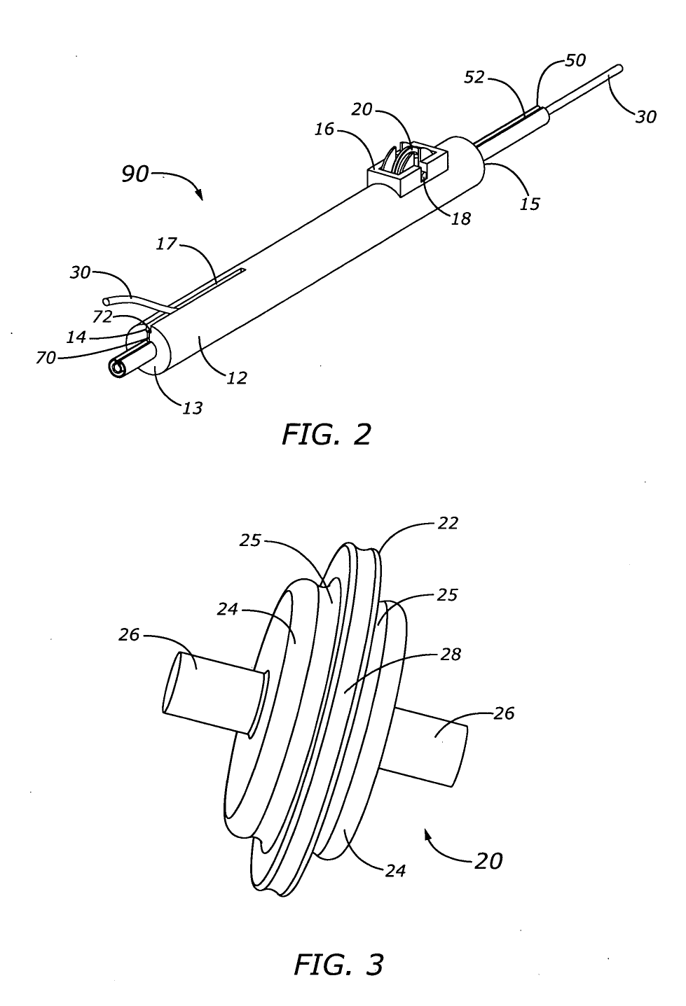

FIG. 2 is a perspective view of the catheter guiding assembly 90 according to an exemplary embodiment of the invention. The assembly includes a housing 12 that is depicted in conjunction with the catheter 50. The housing 12 has a proximal end 13 and a distal end 15, with the distal end 15 being closest to the patient and the proximal end 13 being farthest away from the patient. The catheter 50 travels through a central opening 14 as it is advanced into or retracted from a location within a patient. The catheter's advancement or retraction is manipulated by rotating a wheel 20 that is secured in a wheel receiving body or port 16 that is part of the housing 12. The port 16 is in communication with the central opening 14 and consequently allows the wheel 20 to radially extend into the central opening 14 and frictionally engage with the catheter 50. As the wheel 20 rotates, it pushes a guidewire 30 into a guideway 52 formed in the catheter 50. The catheter guiding assembly 90 includes a receiving slot 17 through which a tool 70 is inserted for backloading the guidewire 30.

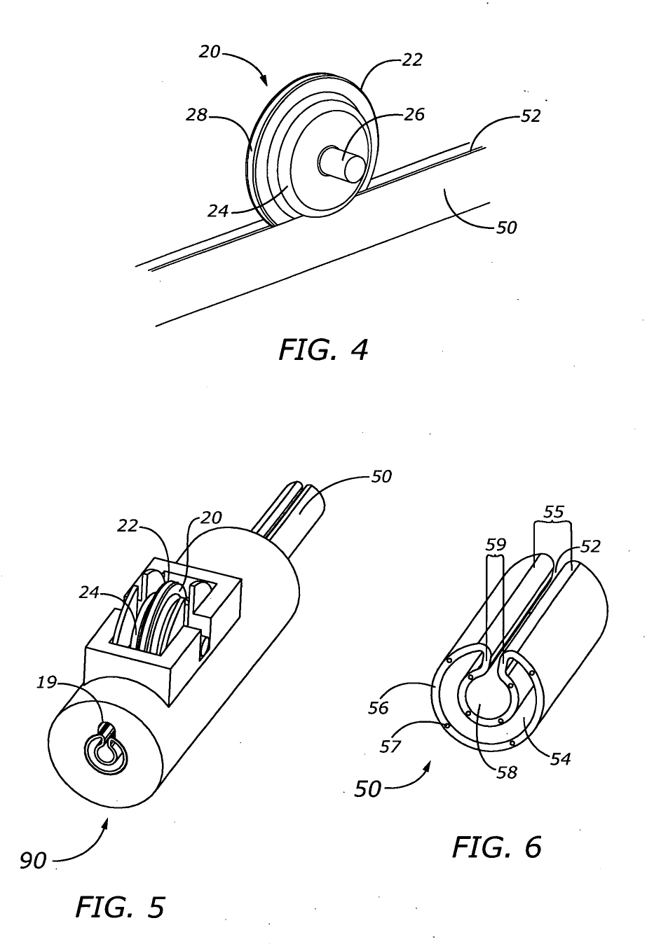

FIG. 3 is a perspective view of the wheel 20 according to one embodiment of the invention. The wheel 20 includes an axle 26 that is receivable into slots 18 formed in the wheel receiving port 16. The entire wheel 20 rotates as the catheter 50 is advanced and retracted. With the axle secured and rotating with little friction in the slots 18, there is little resistance to catheter advancement and retraction. The slots 18 may each include a clip or other device to hold the wheel 20 in place and thereby prevent the wheel from disengaging with the catheter 50. Approximately at the center of the wheel width, a thin, large diameter portion 22 extends radially from two flanking smaller diameter portions 24. The large diameter portion 22 is used to force open the catheter guideway 52, and also to push the guidewire 30 into the catheter guideway 52. The smaller diameter portions 24 are frictionally engaged with the catheter 50 on each side of the catheter guideway 52 and are used, together with the large diameter portion 22, to advance and retract the catheter 50 as the wheel 20 rotates. FIG. 4 is a perspective view of the wheel 20 and the catheter 50 positioned with the large diameter wheel portion 22 in the catheter guideway 52 and the small diameter portions frictionally engaged with the catheter 50. FIG. 5 is a sectional view of the catheter guiding assembly 90 proximal to the wheel 20, with the wheel and catheter 50 in the same arrangement depicted in FIG. 4. In an exemplary embodiment of the invention, the large diameter portion 22 has a concave outer surface 28 to prevent the guidewire 30 from moving laterally as the guidewire 30 is pushed into the catheter guideway 52. Further, the opening 14 in the guiding assembly 90 is continuous with an arched guidewire passageway 19 proximal to the wheel 20 to contain and laterally support the guidewire 30 as it moves between the guiding assembly proximal end 13 and the wheel 20 inside the guiding assembly 90.

The catheter guiding assembly 90, including the wheel 20, is made from blends of polyamides and polyolefins in an exemplary embodiment of the invention. Other exemplary materials include ceramics, metals such as stainless steel, and other polymers such as polyamides and liquid crystal polymers. Lubrication additives such as polyethylene micro-powders, fluoropolymers, silicone-based oils, fluoro-ether oils, molybdenum disulphide, and polyethylene oxide may be included. Reinforcing additives such as nano-clays, graphite, carbon fibers, glass fibers, polyesters, polyketones, polyimides, polysulphones, polyoxymethylenes, polyolefins, cross-linked polyolefins may also be included, along with compatibilizers based on polyolefins, such as grafted polyolefins, ceramics, and metals.

Turning now to the catheter 50 used with the guiding assembly 90, FIG. 6 is a sectional view of the catheter 50, including a nearly coaxially arranged guidewire lumen 58 and inflation lumen 54. Both lumens 54, 58 are formed from a single continuous shaft wall 56 that may be formed from suitable biomedical grade materials such as polyethylene, cross-linked polyethylene, polyolefins, polyamides, blends of polyamides and polyolefins, fluoropolymers, polyesters, polyketones, polyimides, polysulphones, polyoxymethylenes, and compatibilizers based on polyolefins, including grafted polyolefins, and other comparable materials. A lubrication additive may also be used with any polymer and may include polyethylene micro-powders, fluoropolymers, silicone based oils, fluoro-ether oils, molybdenum disulphide and polyethylene oxide. Additionally, a reinforcing additive may be used such as nano-clays, graphite, carbon fibers, glass fibers, and polymeric fibers. The shaft wall 56 that defines the entire inflation lumen 58 is depicted in FIG. 6 as having a substantially uniform thickness, which may simplify the catheter manufacturing process and reduce the associated costs. However, the shaft wall 56 may also be formed with a varying thickness to provide both strength and flexibility to the catheter 50 as needed.

Both the guidewire lumen 58 and the inflation lumen 54 extend from the catheter distal end to the proximal end in an exemplary embodiment of the invention. The generally c-shaped, nearly annular inflation lumen 54 almost entirely surrounds the guidewire lumen 58. A guideway 52 interrupts the inflation lumen's annular shape and prevents the inflation lumen 54 from forming a complete ring. The shaft inner diameter that defines the guidewire lumen 58 is at least 0.015 inch (≈0.38 mm), which is wide enough to permit free movement of contemporary guidewires, which typically have an outer diameter of about 0.014 inch (≈0.36 mm). The shaft has a substantially uniform outer diameter of about 0.039 inch (≈1 mm) in an exemplary embodiment.

Stiffening wires 57 may be incorporated into the lumen walls to strengthen the catheter and maintain its shape. The wires 57 may be incorporated into the catheter 50 in several ways. The wires 57 can be placed in the inflation lumen and attached to the lumen walls, or the shafts can be molded around the wires 57, as illustrated in FIG. 6. Instead of or in addition to stiffening wires 57, a stiffening member in the form of a substantially c-shaped metal strip may be disposed inside the inflation lumen and lining the lumen walls. The stiffening member may have another shape to adapt to the catheter shape and use, but the stiffening member should not impede fluid flow through the inflation lumen.

The guideway 52 is defined by approximately parallel wall segments 59 that are adapted to be flexibly spaced apart to provide transverse access for the guidewire to enter and exit the guidewire lumen 58. When the catheter 100 is tightened in a Y-adapter, the wall segments 59 will rest flatly against each other, thereby preventing back-bleed and also sealing the guideway 52 and reducing or eliminating any clearance around the guidewire.

In an exemplary embodiment of the invention, a guidewire entrance 55 with a gradually curved contour joins the guideway 52 and the catheter outer diameter. The guidewire entrance 55 further prevents the guidewire 30 from moving laterally and thereby impeding its insertion into the guideway 52. In a further exemplary embodiment, the wheel smaller diameter portions 24 that flank the large diameter portion 22 have concave surfaces 25 that are rounded to match the catheter's curved guidewire entrance 55 and thereby improve the frictional engagement between the wheel 20 and the catheter 50.

The operation of the catheter 50 and the guiding assembly 90 will now be described. After the guidewire 30 is inserted into the patient, the guidewire 30 can be combined with the catheter by backloading the catheter 50 onto the guidewire 30. Using a backloading process, the guidewire proximal end is threaded through the catheter guidewire lumen 58 until the guidewire proximal end either exits the catheter proximal end or reaches a proximal position of the catheter 50 relative to the wheel 20, at which point the guidewire 30 can be removed from the guidewire lumen 58 using a suitable tool, embodiments of which are described below. With the guidewire 30 removed from the guidewire lumen, the guidewire 30 can be held in place as the catheter 50 is advanced through the guiding assembly 90 and into the patient. As the catheter 50 advances, the wheel pushes the stationary guidewire 30 into the guidewire lumen 58.

If a guidewire exchange is required, the physician or other user can simply pull out the guidewire 30 with the catheter 50 remaining stationary. A new guidewire can be loaded into the proximal guidewire lumen 58 and threaded through the catheter 50. The distal portion of the guidewire 30 is typically flexible and difficult to insert into the guidewire lumen 58. Consequently, the distal end of the new guidewire 30 can be positioned above the guideway 59, and then, with the catheter 50 and guidewire 30 kept stationary, the guiding assembly 90 can be moved proximally over the guidewire until the wheel 20 pushes the guidewire 30 into the guidewire lumen 58. Once the guidewire flexible distal portion is inside the guidewire lumen 58, the guidewire can be advanced by pushing the guidewire 30 through the catheter 50, and the guiding assembly 90 can be returned to a position closer to the patient if necessary.

If a catheter exchange is required, the physician or other user holds the guidewire 30 in place and retracts the catheter proximally by rotating the wheel 20. After the catheter is removed, the replacement catheter is installed using the process described above.

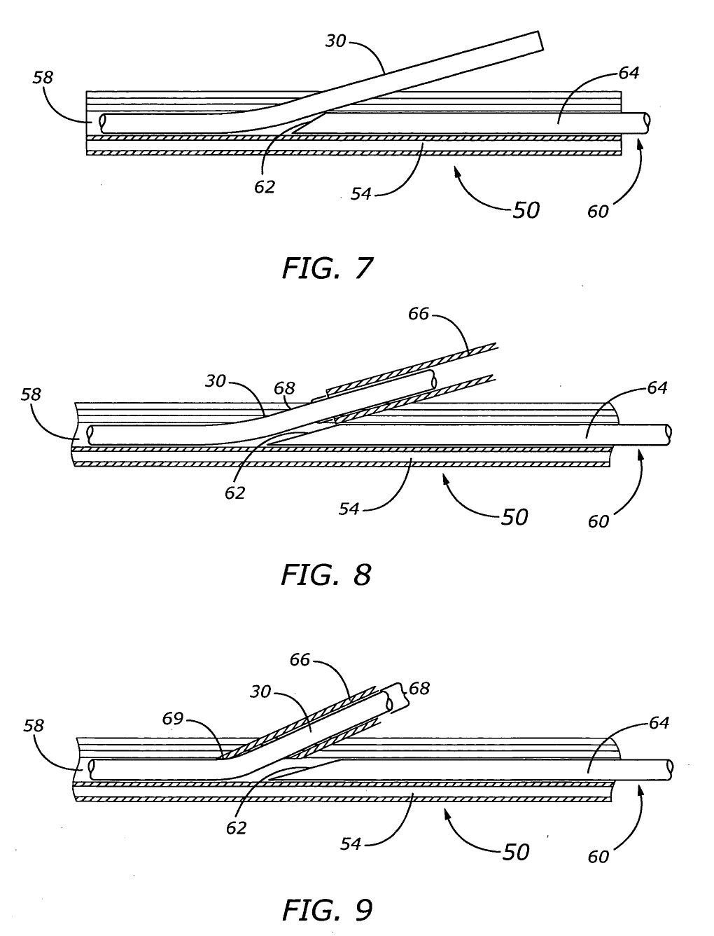

As mentioned above, it is typically necessary to remove at least a portion of the guidewire 30 from the proximal catheter shaft. A suitable tool may be utilized in order to quickly remove the guidewire 30. FIG. 7 is a sectional view of the catheter 50 with the guidewire 30 partially disposed in the guidewire lumen 58, and partially removed from the guidewire lumen 58 using a guidewire removal tool 60 according to one embodiment of the invention. The tool 60 includes a substantially cylindrical main body 64, and a chamfered leading edge 62 that can be a flat beveled surface, a concave or grooved surface, a v-shaped surface, or other suitable surface that can raise the guidewire 30 out of the guidewire lumen 58.

The main body 64 is sized to have a diameter that approximates that of the guidewire 30 so the tool 60 can effectively raise the guidewire 30 and force it out from the guidewire lumen 58. The main body 64 may have a slightly larger diameter than the guidewire 30 as long as the diameter is not larger than the guidewire lumen inner diameter or large enough to create enough friction to prevent the tool 60 from extending a significant distance into the guidewire lumen 58. In an exemplary embodiment of the invention, the main body 64 has a substantially uniform diameter ranging between about 0.016 inch and about 0.017 inch, and is used with a guidewire 30 that has a diameter of about 0.017 inch.

In an exemplary embodiment of the invention, the tool 60 is a rigid body and is formed entirely from a metallic material. The strong and rigid metal provides the advantages of ease in placing the tool 60 in a desired location and thereafter manipulating the tool to raise the guidewire 60. If the tool will be distally extended a significant distance into the catheter 50 then the metallic material can be somewhat bendable although the tool 60 should be rigid enough to easily manipulate the leading edge when holding the tool 60 from a from an upstream or proximal point. The tool 60 can be formed from a wire mandrel and can be as long or as rigid as necessary to perform the desired function. One advantage of a substantially elongated and rigid tool 60 is its ability to perform a stiffening function for at least some catheter length that is proximal to the catheter guiding assembly 100. For instance, without the tool 60 inserted into the guidewire lumen 58, the catheter is advanced by grasping the catheter 50 a short distance from the guiding assembly 90 and pushing the catheter into the guiding assembly. With a long and rigid tool 60 inserted into the guidewire lumen 58 at or near the proximal end 13 of the guiding assembly 90, the advancing force can be applied to the catheter 50 much farther away from the guiding assembly 90, and consequently a longer catheter length can be advanced with each push.

Referring now to FIG. 8, a sectional view of another embodiment of a guidewire removal tool is depicted. According to this embodiment, the tool 60 is used in conjunction with a guideway opening tube 66 into which the guidewire 30 is backloaded as the guidewire 30 is forced from the guidewire lumen 58. The tube 66 is formed from metal, plastic, or other sufficiently rigid material to enter and widen the guideway 52 through which the guidewire 30 enters the guidewire lumen 58, while maintaining a substantially uniform inner diameter 68 through which the guidewire 30 can freely advance and retract. With the tool 60 lifting the guidewire 30 and the tube 66 widening the guideway to receive the guidewire 30 from the guidewire lumen 58, the length of the guidewire 30 will be continuously received into the tube 66 as the catheter is retracted. The tube 66 can also facilitate replacing the guidewire 30 into the guidewire lumen 58 by advancing the catheter 50 into the patient or by simply pushing the guidewire 30 through the tube 66 and into the catheter 50.

The tube 66 and tool 60 are used alone, without any supporting apparatus in an exemplary embodiment of the invention. However, the tube 66 and tool 60 can be used in conjunction with the catheter guiding assembly in another embodiment. Returning to FIG. 2, the catheter guiding assembly 100 can include a receiving slot 17 through which the tube 66 can be inserted and laterally supported to maintain its position in the guideway 52 as a practitioner backloads the guidewire 30. In either embodiment, the tube 66 can be further supported by resting it on the tool 60 within the guidewire lumen 58 as illustrated in FIG. 8.

To aid in inserting the tube 66 into the guideway 52, the tube 66 can have a beveled leading edge 69. FIG. 9 is a sectional view of the catheter 50 receiving the guidewire removal tool 60 With the leading edge 69 being beveled at an angle, the tube 66 will have a pointed tip that opens the guideway 52 and leads the tube 66 into the guidewire lumen 58 to receive the guidewire 30 and lead it out of the catheter 50.

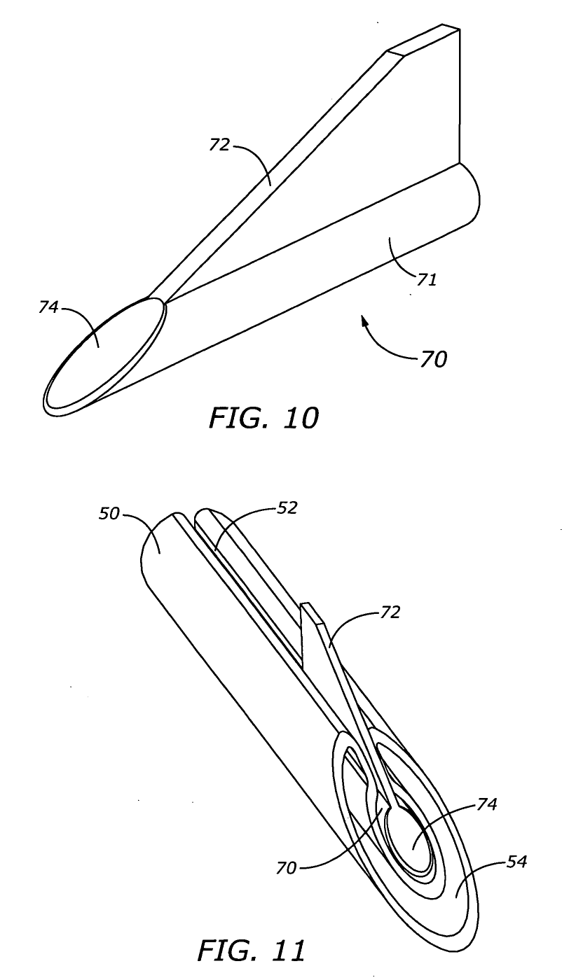

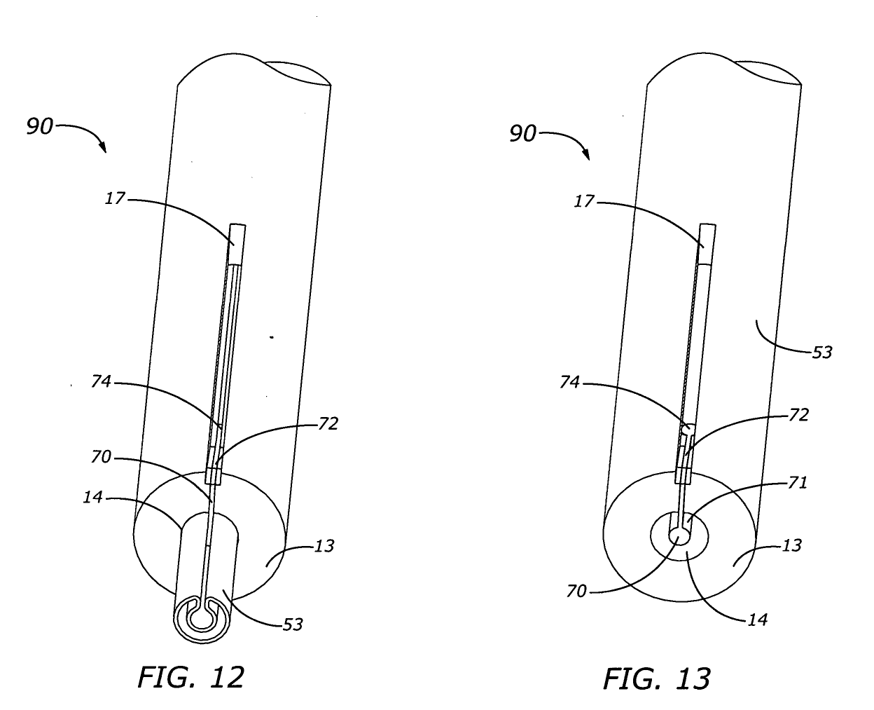

FIG. 10 is a perspective view of another exemplary embodiment of a guidewire removal tool 70. The tool 70 includes a substantially cylindrical main body 71, and a chamfered leading edge 74 that can be a flat beveled surface, a concave or grooved surface, a v-shaped surface, or other suitable surface that can raise the guidewire 30 out of the guidewire lumen 58, and can have the characteristics as the cylindrical main body 64 described above. The tool 70 also includes a fin 72 that interacts with the guidewire lumen 58 in the manner illustrated in FIG. 11, which is a sectional view of the catheter 50 with the guidewire removal tool 70 inserted in the guidewire lumen 58. The fin 72 can protrude through the guiding assembly receiving slot 17 as the tool 70 is advanced into the guiding assembly 90 from the proximal end 13. In an exemplary embodiment of the invention, the fin 72 is stabilized by the slot 17, which also fixes the tool 70 in the guiding assembly 90. In another exemplary embodiment, the fin 72 provides a handle for the physician or other user to push the tool into the catheter guiding assembly 90. More particularly, the fin 72 protrudes from the receiving slot 17 and enables the physician or other user to move the tool 70 close to the wheel 20 and thereby remove the guidewire 30 from the guidewire lumen 58 near the guiding assembly distal end 15.

FIG. 12 is a perspective view of the guide member distal end 13 in cooperation with the guidewire removal tool 70 and the proximal shaft 53. FIG. 13 is a perspective view of the guide member distal end 13 and the guidewire removal tool 70, with the proximal shaft removed to more clearly illustrate the relationship between the guide member and the guidewire removal tool 70.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

What is claimed is:

1. A catheter guiding assembly, comprising:
a catheter, comprising:
an elongate shaft having an exterior surface, a proximal end, and a distal end,
a first lumen extending through the shaft from the shaft proximal end to the shaft distal end, and sized to receive a guidewire, and
a guideway extending from the shaft proximal end to the shaft distal end, and enabling transverse access from the shaft exterior surface to the first lumen;
a catheter advancing and retracting apparatus, comprising:
an elongate housing having a proximal end and a distal end,
an opening formed through the housing from the housing proximal end to the housing distal end and adapted to receive the catheter, and
a linear slot formed in the housing proximal end and adapted to be aligned with a portion of the catheter guideway; and
a guidewire removal tool, comprising:
a substantially cylindrical main body sized to be receivable by the first lumen and having a chamfered leading edge adapted to raise the guidewire out of the first lumen through the guideway.

2. The catheter guiding assembly according to claim 1, wherein the chamfered leading edge has a flat, beveled surface.

3. The catheter guiding assembly according to claim 1, wherein the chamfered leading edge has a concave surface.

4. The catheter guiding assembly according to claim 1, wherein the chamfered leading edge has a v-shaped surface.

5. The catheter guiding assembly according to claim 1, wherein the substantially cylindrical main body is a metallic material.

6. The catheter guiding assembly according to claim 1, wherein the guidewire removal tool further comprises:
a guideway opening tube formed from a rigid material and adapted to be receivable through the guideway into the first lumen to remove the guidewire from the first lumen.

7. The catheter guiding assembly according to claim 6, wherein the guideway opening tube is adapted to be further receivable through the linear slot formed in the housing proximal end.

8. The catheter guiding assembly according to claim 6, wherein the guideway opening tube has a beveled leading edge.

9. The catheter guiding assembly according to claim 8, wherein the leading edge has a pointed tip.

10. The catheter guiding assembly according to claim 1, further comprising a fin formed on the substantially cylindrical main body and adapted to extend out of the first lumen through the guideway when the main body is received by the first lumen.

11. The catheter guiding assembly according to claim 10, wherein the fin is further adapted to extend out of the linear slot formed in the housing proximal end.

12. The catheter guiding assembly according to claim 1, wherein the catheter further comprises a second lumen extending through the shaft from the shaft proximal end to the shaft distal end, and having a nearly annular cross sectional area that almost entirely surrounds the first lumen.

13. The catheter guiding assembly according to claim 12, wherein the first lumen and the second lumen are both formed from a single wall having a substantially uniform thickness.

14. The catheter guiding assembly according to claim 13, wherein the single wall includes two approximately parallel segments that together define the catheter guideway.

15. The catheter guiding assembly according to claim 14, wherein the two parallel segments are adapted to be flexibly forced apart.

16. An apparatus for advancing and retracting a guidewire and a catheter having a lumen, an exterior surface, and a guideway that enables transverse access from the catheter exterior surface to the lumen in a patient, the apparatus comprising:
an elongate housing having a proximal end and a distal end;
a opening formed through the housing from the proximal end to the distal end and adapted to house the catheter and guidewire;
a linear slot formed in the housing and extending from the proximal end; and
a guidewire removal tool having a substantially cylindrical main body sized to be receivable by the opening at the housing proximal end, and having a chamfered leading edge adapted to raise the guidewire out of the catheter through the guideway.

17. The apparatus according to claim 16, wherein the chamfered leading edge has a flat, beveled surface.

18. The apparatus according to claim 16, wherein the chamfered leading edge has a concave surface.

19. The apparatus according to claim 16, wherein the chamfered leading edge has a v-shaped surface.

20. The apparatus according to claim 16, wherein the substantially cylindrical main body is a metallic material.

21. The apparatus according to claim 16, wherein the guidewire removal tool further comprises:
a guideway opening tube formed from a rigid material and adapted to be receivable through the guideway into the first lumen to remove the guidewire from the first lumen.

22. The apparatus according to claim 21, wherein the guideway opening tube is adapted to be further receivable through the linear slot formed in the housing proximal end.

23. The apparatus according to claim 21, wherein the guideway opening tube has a beveled leading edge.

24. The apparatus according to claim 23, wherein the leading edge has a pointed tip.

25. The apparatus according to claim 16, further comprising a fin formed on the substantially cylindrical main body and adapted to extend out of the first lumen through the guideway when the main body is received by the first lumen.

26. The apparatus according to claim 25, wherein the fin is further adapted to extend out of the linear slot formed in the housing proximal end.

27. A tool for removing a guidewire from a catheter having a lumen, an exterior surface, and a guideway that enables transverse access from the catheter exterior surface to the lumen, the tool comprising:
a substantially cylindrical main body sized to be receivable by the lumen and having a chamfered leading edge adapted to raise the guidewire out of the first lumen through the guideway.

28. The tool according to claim 27, wherein the chamfered leading edge has a flat, beveled surface.

29. The tool according to claim 27, wherein the chamfered leading edge has a concave surface.

30. The tool according to claim 27, wherein the chamfered leading edge has a v-shaped surface.

31. The tool according to claim 27, wherein the substantially cylindrical main body is a metallic material.

32. The tool according to claim 27, wherein the guidewire removal tool further comprises:
a guideway opening tube formed from a rigid material and adapted to be receivable through the guideway into the first lumen to remove the guidewire from the first lumen.

33. The tool according to claim 32, wherein the guideway opening tube has a beveled leading edge.

34. The tool according to claim 33, wherein the leading edge has a pointed tip.

35. The tool according to claim 27, further comprising a fin formed on the substantially cylindrical main body and adapted to extend out of the first lumen through the guideway when the main body is received by the first lumen.
